(19) United States
(12) Patent Application Publication
(10) Pub. No.: US 2008/0171001 A1
(43) Pub. Date: Jul. 17, 2008

(54) MINERALIZATION TOOTHPASTE

(76) Inventors: Emil E. Engelman, Doylestown, PA (US); Robert J. Gambogi, Hillsborough, NJ (US); Deepak Sharma, Flemington, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/972,693

(22) Filed: Jan. 11, 2008

Related U.S. Application Data
(60) Provisional application No. 60/884,738, filed on Jan. 12, 2007.

(51) Int. Cl. A61K 8/66; A61K 8/55; A61Q 11/00

(52) U.S. Cl. 424/50; 424/57

(57) ABSTRACT

The present invention is directed to a tooth cleaning and mineralizing composition including an abrasive composition in an amount sufficient to provide the tooth cleaning and mineralizing composition with a relative dental abrasion of from 100 to 250; and a stabilized tooth mineralizing mineral composition.

### FIELD OF THE INVENTION

This invention relates generally to toothpaste or dentifrice compositions that provide mineralization of tooth surfaces.

### BACKGROUND OF THE INVENTION

Dentifrices, especially toothpastes, typically contain abrasive materials for cleaning teeth. The relative abrasive nature, or abrasivity, of the compositions may be adjusted by the type of abrasive and the amount of abrasive in the composition. Abrasivity may be quantified by several methods, for example the Hefferen abrasivity procedure described in ISO 11609, “Dentistry-Toothpastes-Requirements, test methods and marking”. Generally, the higher the abrasivity, the better the tooth cleaning effect.

As people age, their teeth tend to lose surface minerals due to tooth wear and chemical erosion. This phenomenon is particularly apparent along the gum line, where tooth sensitivity may be experienced. Several products have been developed to mineralize the surface of teeth. These products typically contain calcium and phosphate compounds, which can deposit on the teeth and grow through crystallization. The minerals may be encapsulated in or bound to poly(amino acids), (such as polylysine, polyglutamic acid, etc), polypeptides or protein (such as casein phosphopeptides and gelatin), poly electrolytes (such as Tris(hydroxymethyl) amino methane (TRIS) and 2-(N-morpholino) ethanesulfonic acid (MES)), and polymers. Other examples include stabilized amorphous calcium phosphate, amorphous calcium phosphate and hydroxyapatite. These materials may be unstable in the sense that they may agglomerate or prematurely crystallize out of solution. For these reasons, surface stabilized mineralization compositions have been developed.

It is desirable to provide one composition that cleans teeth effectively and mineralizes the teeth simultaneously. Unfortunately, higher abrasivity toothpastes are likely to prevent minerals in a dental mineralization composition from depositing on the teeth and growing crystals. The present invention overcomes this problem.

### SUMMARY OF THE INVENTION

The present invention is directed to a high abrasivity toothpaste composition that mineralizes teeth and that contains at least one abrasive composition in an amount sufficient to provide a relative dental abrasion of from 100 to 250 and a stabilized tooth mineralizing mineral composition. As used herein, the term high abrasivity means a relative dental abrasion between 100 and 250 as measured by the Hefferen abrasivity procedure.

### DETAILED DESCRIPTION OF THE INVENTION

The compositions of the present invention include at least one abrasive composition in an amount sufficient to provide a relative dental abrasion of from 100 to 250. Suitable abrasive compositions include, but are not limited to, anhydrous dicalcium phosphate, dicalcium phosphate dehydrate, calcium carbonate, calcium pyrophosphate, sodium bicarbonate, silica, alumina, aluminosilicates and combinations thereof. The amount of abrasive composition may range from 1% to 60% by weight, based on the total weight of the composition.

The compositions of the present invention include a stabilized tooth mineralizing mineral composition. As used herein, stabilized tooth mineralizing mineral compositions means a composition that contains a source of calcium and phosphate and that has been stabilized with a proteinaceous material or a synthetic polymeric electrolyte that has an affinity for the mineral surface, resulting in a more stable form of mineral that increases the bioavailability of mineralizing ions, e.g. calcium and phosphate, in the oral cavity. Thus, the compositions are more likely to provide a mineralization benefit upon use.

The compositions of the invention may include from 5% to 70% by weight of at least one carrier. Suitable carriers include, but are not limited to, glycerin, propylene glycol, sorbitol, and combinations thereof. Chelating agents, such as sodium ethylene diamine tetracetic acid (EDTA) or sodium acid pyrophosphate may also be utilized in the compositions of the present invention. Anti-caries agents, such as a fluoride agent, including, but not limited to, stannous fluoride, sodium fluoride, and sodium monoflourophosphate, as well as other therapeutic agents, may be useful in the compositions of the present invention at levels from 0.1 to 1.14% by weight, based on the total weight of the composition. The compositions of the present invention may further include binders and thickening agents, including, but not limited to, colloidal silica, carrageenan, xanthan gum, methyl cellulose, carbopol, and combinations thereof at levels ranging from 0.5% to 10% by weight, based on the total weight of the composition. Surfactants, such as sodium lauryl sulfate, betaines, sodium lauryl sarcosinate, ethylene oxide/propylene oxide copolymers, sorbitan derivatives, and the like may also be useful in the compositions of the present invention at levels ranging from 0.1% to 8% by weight, based on the total weight of the composition. The compositions of the present invention may further include from 0.1% to 7% by weight flavors, and from 1% to 10% by weight whiteners selected from the group consisting of hydrogen peroxide, carbamide hydrogen peroxide, protease enzymes, polyphosphates, and pyrophosphates. Tartar control agents and antiplaque/antigingivitis agents may also be useful in the compositions of the present invention at levels known in the art.

One example of a stabilized tooth mineralization mineral composition is an apatite stabilized by collagen, gelatin or other synthetic poly(amino acid) materials. A stabilizing protein may be reacted in solution to encapsulate the minerals. Alternatively, the materials may be combined to associate the protein with the minerals.

The compositions of the present invention include stabilized tooth mineralizing mineral compositions that release, for example, calcium and phosphate on application. One example of a stabilized mineralization composition is a casein phosphopeptide—amorphous calcium phosphate composition. PCT Application WO 98/40406, hereby incorporated by reference, discloses stabilized tooth mineralizing mineral compositions and methods for preparing them. Moreover, synthetic peptides can be used to develop bio-available amorphous calcium phosphate salts. Materials synthesized with D-amino acids can be utilized to render them protease-resistant. This enables compositions with enzymes (proteases) that can be used to modify the proteins on oral surfaces for an improved health or aesthetic benefit without inducing any destabilization of the amorphous calcium phosphate. Similarly, D-aminio acid proteins can be used to stabilize hydroxyapatite and similar minerals that can be used in formulations with proteases.

Suitable sources of minerals include, but are not limited to, tricalcium phosphate, dicalcium phosphate, calcium dihydrogen phosphate, calcium pyrophosphate, hydroxyapatite, fluoroapatite and combinations thereof. The protein and the calcium phosphate source are typically combined at ratios of 10:1 to 0.1:10, depending on the surface area of the calcium phosphate moiety.

The amount of stabilized tooth mineralizing minerals in the compositions of the present invention may range from 0.1% to 10% by weight, based on the total weight of the composition.

The compositions of the present invention may further include a proteolytic enzyme. Examples of suitable proteolytic enzymes include, but are not limited to, serine proteases, threonine proteases, cysteine proteases, aspartic acid proteases, metalloproteases and glutamic acid proteases. Alternatively, a proteolytic enzyme may be mixed with the compositions of the present invention upon use, such as through the use of a dual chambered tube. Proteolytic enzymes that may be used in the present invention include papain. As used herein, papain refers to the crystalline proteolytic enzyme rather than the crude dried latex. It is a preparation from commercial dried papaya latex. According to the Merck Index, the papain molecule consists of one folded polypeptide chain of 212 residues with a molecular weight of about 23,400. If papain is used, it may be incorporated in the amount of about 0.05 to 7.5% by weight. Formulations will be developed to maintain the papain activity, as determined by the Milk Clot Assay Test of the Biddle-Sawyer Group. (See J. Biol. Chem., Volume 121, pages 737-745, (1937)). Traditionally papain activities of raw materials can be on the order of 800 MCU/mg. If papain having a different activity were to be used, it would be adjusted in an amount to correspond to effective quantities.

It is theorized that surface adsorbed proteins or peptides may control the crystal growth or integrity of calcium phosphates. Proteolytic enzymes will degrade or digest these proteins or peptides and the crystal growth properties of the calcium phosphate or the release properties of these moieties will be modified.

The activation of stabilized amorphous calcium phosphate materials that have been made protease-resistant using D-amino acid peptides may be, and in one embodiment, preferably is, carried out on application to the treatment area of interest, for example the surface of teeth or bone. The enzyme and stabilized calcium phosphate may be kept separate until time of activation through multi-compartment delivery devices or through encapsulation of either enzyme or stabilized calcium phosphate.

Additional ingredients that may be incorporated in the compositions of the present invention are antibacterial agents including noncationic antibacterial agents such as halogenated diphenyl ethers such as 2′,4,4′-trichloro-2-hydroxy-diphenyl ether (Triclosan) and phenolic compounds including phenols, and their homologs, mono-and polyalkyl and aromatic halophenols, resorcinol and its derivatives, bisphenolic compounds and halogenated salicylanilides. Examples of other antibacterial agents that may be included in the compositions include chlorhexidine; copper- and zinc-salts such as zinc chloride, zinc citrate and sodium zinc citrate; sanguinarine extract, and metronidazole; quaternary ammonium compounds such as cetylpyridinium chloride; bis-guanides such as chlorhexidine digluconate, hexetidine, octenidine and alexidine; essential oils; stannous fluoride, and combinations thereof. The antibacterial agent may be present in the composition in an effective antiplaque amount, typically 0.01% to 5% by weight.

Anti-inflammatory agents such as ibuprofen, flurbiprofen, aspirin, and indomethacin etc. may also be included in the abrasive composition. Agents useful in the treatment of dentin hypersensitivity also may be used in the present invention. Such agents include, without limitation, stannous fluoride, potassium salts such as potassium citrate, potassium chloride, amorphous calcium phosphate, potassium sulfate, potassium tartrate, oxalate salts and potassium nitrate.

The compositions used in the present invention may be prepared by conventional methods. Containers used to house the compositions may be of any type conventionally used, and include dual chamber products currently known and sold. The compositions of the present invention may also be useful in chewing gums, tablets, and alternate chewable forms.

An example is set forth below for illustrative purposes. The invention should not be construed to be limited to the details thereof.

### EXAMPLE

A composition is prepared according to the invention by combining the materials in Table 1 in a vessel and stirring. One composition is made with papain.

A second composition is prepared without papain (substituting water for it). In a third experiment, the composition without papain above is placed in one chamber of a commercially available dual chamber dispensing tube. A solution of papain is placed in the other chamber of the dual chamber dispensing tube.

TABLE 1

| Ingredient | Weight Percent |
|---|---|
| USP Sorbitol Solution 70% | 45.0 |
| Glycerin, USP | 10.0 |
| Water | 21.757 |
| Hydrated Silica Abrasive | 17.0 |
| Flavor | 1.2 |
| Sodium Lauryl Sulfate | 1.0 |
| Papain | 0.8 |
| Carbopol 974P NF | 0.5 |
| Xanthan Gum | 0.5 |
| Titanium Dioxide | 0.5 |
| Carboxymethylcellulose | 0.5 |
| Sodium Fluoride | 0.243 |
| Stabilized calcium phosphate | 1.0 |

The compositions are combined in a vessel and stirred until homogenous. The compositions are used to brush teeth. The composition without papain cleans and mineralizes the teeth effectively. The composition with papain is more effective than the composition without papain at cleaning and mineralizing the teeth. The composition of the present invention is most effective when papain is mixed with the composition upon use, for example through the use of a dual tube dispenser with the composition above in one chamber and papain in the other chamber.

We claim:

1. A tooth cleaning and mineralizing composition, comprising:
an abrasive composition in an amount sufficient to provide said tooth cleaning and mineralizing composition with a relative dental abrasion of from 100 to 250; and
a stabilized tooth mineralizing mineral composition.

2. The tooth cleaning and mineralizing composition according to claim 1 comprising from 1 to 60 percent by weight of said abrasive composition.

3. The tooth cleaning and mineralizing composition according to claim 2 comprising from 0.1 to 10 percent by weight of said stabilized tooth mineralizing mineral composition.

4. The tooth cleaning and mineralizing composition according to claim 3 wherein said stabilized tooth mineralizing mineral composition is selected from the group consisting of a casein phosphopeptide amorphous calcium phosphate and stabilized hydroxyapatite.

5. The tooth cleaning and mineralizing composition according to claim 4 wherein said abrasive composition comprises an abrasive selected from the group consisting of anhydrous dicalcium phosphate, calcium carbonate, calcium pyrophosphate, sodium bicarbonate, hydrated silica and alumina.

6. The tooth cleaning and mineralizing composition according to claim 3 wherein said tooth mineralizing mineral is protease-resistant.

7. The tooth cleaning and mineralizing composition according to claim 1 further comprising a proteolytic enzyme.

8. The tooth cleaning and mineralizing composition according to claim 5 comprising from about 0.05 to 7.5 percent by weight of a proteolytic enzyme selected from the group consisting of serine proteases, threonine proteases, cysteine proteases, aspartic acid proteases, metalloproteases and glutamic acid proteases.

9. The tooth cleaning and mineralizing composition according to claim 5 comprising about 17 percent by weight of said abrasive composition, about 1 percent by weight of said stabilized tooth mineralizing mineral composition and about 0.8 percent by weight of said proteolytic enzyme.

10. The tooth cleaning and mineralizing composition according to claim 9 wherein said abrasive composition comprises silica, said stabilized mineralizing mineral composition comprises casein phosphopeptide amorphous calcium phosphate and said proteolytic enzyme comprises papain.

11. The tooth cleaning and mineralizing composition according to claim 1 further comprising a whitener selected from the group consisting of hydrogen peroxide, carbamide hydrogen peroxide, protease enzymes, polyphosphates and pyrophosphates.
